SEQUENCE LISTING (15 sequences)

SEQ ID NO: 1 - DNA, 1398 bp, Candida tropicalis
atgcaattgtcaattgtttgcttctttcttttaacatatgttaatgctgctgtcatcacattgacaaaaccatcggatgatgatttctacattccacaagatggttttgaagatgctgaaccaggtgagattttgaagatccgaaagacccccaacaagcttagtggtttatttttcccaatagatgtcaaaaatagttggcaattgttagtacgctctactgattcctttggaaatgccactgctattgttgctactatcatagaaccattcaattctgatccttccaaagtggtatcctatcaaacttttgaagattcggccaatattaactgttcaccttcctatgggatgcaatggggtgcttctatttccactgttgcgacacagattgatatgtcatttatggtcccattgttaaacaatggttactttttggtatctccagattatgaaggtcctaaatcaactttcactgtgggtagacaatcgggacatggtactttggactcaatcagagccattttacaatctggaaattttactggtgttgatgaagatgcacaggttgctatgtggggttattctggtggttctttggcatcaggttgggctgctgctttgcaaccacattatgcacctgaattggaagataatttgattggtgctgcccttggtggatttgtaaccaatattacagctactgctgaagctacagatggaaagttattggctggcttagtacctattgctttgaatggattgggtaatgaatataatgatttccgtgaaatattgtactctgaagtcaaagaaggtggaagagataaattggctgatgggttaaatcattgtatgattcctggtatcattcgctttgcctttagccaatttctagcaggtaaaaatagactattccctaatggttacggtttgctcgacgacccaatagttaacagaacaatccaggaaaacaatcttatgagtgtctccaaggagtacattcctaaaataccgttatttgtttatcatggcacattggatgcagtggttccaattgtcaatgtcaagaaaacctatgaaagatggtgtgattggggtattgaatcttttgaatttgctgaagacttgttgaatggtcacatttctgaaactttagttggtgcaccagctgcgttgacatggttggaaagaagattcgctggtttggatccagtcaaaggatgccaacatactgcacgaatgatgaacttcttatacccaaatatctcaagtgctactagtgattactttacaggattgtatgatgctcttcaaggcaccccattgggaccaggattgaacaccgacaactttacacttaatggattgctaggaaccttgggtgatattttctag

SEQ ID NO: 2 - DNA, 1554 bp, Candida tropicalis
atgatcgaacaagttgttgaatactggtacgtggtcttaccattggtatttatccttcataaagtatttgacatgtggcacactcgtcggttgatgaagcaattgggcgctgctcctgtcacaaaccaattacacgacaatttttttggtattatcaacggatggaaagcacttaagttcaagaaagaaggtagagctcaagaatataatgattataaatttgccaattcgaaaattccaagtgtgggtacttatgttagtaccatctttggaacaaagctcctcgtcacaaaagatccggagaatatcaaagctttattagcaacccaattcagtgatttttccttgggtaagaggcatacacttttcaaaccattattaggtgacggtattttcactttggatggagaaggttggaaacatagtcgagctatgttgagaccacagtttgcaagagaacaagttgcccatgtcacttctttagagccacatttccaattgttgaaaaaacatatcctcaagaacaaaggaggtttttttgatatccaggaattgtttttccgattcactgttgattcagctactgagtttttgtttggtgaatcagtacactctttgaaggatgaaacaattgggtataaccaagatgatatcgactttgttggtagaaaggattttgcggaatcgttcaacaaggcacaagagtatcttgctattagaactttggtgcaagatttttattatcttgtaaacaatcaggaattcagagattgcaataaactggtacacaagtttaccaactactatgtccaaagagcattggatgccactcctgaagagcttgaaaagcaaagtggatatgttttcttgtatgaattggttaaacaaaccagagaccctaatgtattgagagatcaatcattgaacatcttattagctggtagagataccactgctgggttgttgtcatttgcggtatttgaacttgctaggaatccacatatttgggccaaattaagagaagatgttgaatcccaatttggtcttggtgaagaatctcgcattgaagagattacctttgaaagtttaaaacgatgtgaatacttgaaagctttccttaacgaaacattacgtgtttatccaagtgttccaagaaatttcagaattgctaccaaaaacaccactttaccaagaggtggtggttcagacggcaattctcctgttttggtcaaaaagggcgaggctgtttcatatggtataaattctactcacttagatcctgtctattatggtgacgatgctgcagaatttagaccagaaagatggaacgagccatcaacaagaaaattgggatgggcatatttaccgttcaacggaggcccaagaatttgtttaggtcaacaatttgctttaaccgaagcgggttatgtattggttagattggcccaaagttttgacaccttggaattgaagccaccagttgtgtatccaccaaagagattaacaaacttgactatgtctttacaagacggaactattgtcaagatcgattag

SEQ ID NO: 3 - DNA, 2043 bp, Candida tropicalis
atggcattagataagttagatttatatgttattataacattggtggttgcaattgcagcttattttgcaaagaatcagtttcttgaccaacaacaagataccgggttccttaatactgatagtggagatggtaattcaagagatatcttacaagctttgaagaagaacaataaaaatacgttattattatttggatcccaaacaggtacagcagaagattatgccaacaaattgtcaagagaattgcattcaagatttggtttgaaaaccatggttgctgatttcgctgattatgatttcgaaaacttcggagatattactgaagatatcttggttttctttattgttgctacttatggtgaaggtgaaccaaccgataatgctgacgaatttcacacttggttgactgaagaagctgacaccttgagtactttgaaatatactgtttttggtttgggtaattcaacttatgaattcttcaatgctattggtagaaaatttgacagattgttgggagaaaaaggtggtgacagatttgctgaatacggtgaaggtgacgatggtactggtactttagatgaagatttcttggcctggaaggataacgtgtttgattccttaaagaatgatttgaattttgaagaaaaagagttgaaatacgaaccaaatgttaaattgactgaaagagatgatttatctggcaatgatccagaagtctccttgggtgaaccaaatgtcaaatacattaaatctgaaggtgttgacttaactaaaggtccatttgatcatactcatccatttttggctagaattgttaaaactaaagaattgtttacttctgaagacagacattgtgttcatgttgaatttgatatttctgaatcaaacttgaaatataccaccggtgatcatcttgcaatctggccatctaactctgatgaaaacattaagcaatttgccaaatgttttggtttagaagacaaacttgatactgttattgaattgaaagctttggattccacttattccatcccattccctaatccaatcacttatggagctgttattagacaccatttggaaatttcaggtcctgtttctagacaatttttcttatctattgctggatttgcccctgatgaagaaactaaaaagtcatttactagaattggtggtgataagcaagaatttgctagtaaagtcacccgtagaaaattcaacattgccgatgctttattatttgcttccaacaacagaccatggtccgatgttccattcgaattccttattgaaaatgtccaacacttaactcctcgttattactccatttcttcttcctcattaagtgaaaagcaaaccattaatgttactgctgttgttgaagccgaagaagaagctgatggaagaccagttactggtgttgtcaccaacttgttgaagaatattgaaattgaacaaaacaaaactggtgaaaccccaatggttcattatgatttgaatggtccaagaggcaaatttagcaagttcagattgccagttcacgttagaagatctaatttcaaattaccaaagaatagcactaccccagttattttgattggtccaggtaccggtgttgcaccattgagaggttttgttagagaaagagttcaacaagttaaaaatggtgttaatgttggtaagactgtattgttttatggatgtagaaattccgaacaagatttcttgtacaaacaagaatggagtgaatatgcctcagtattgggagaaaatttcgaaatgtttaatgccttctcaagacaagatccaactaagaaagtttatgttcaagataagattttagaaaatagtgctcttgttgatgagttattatctagtggagcaattatttatgtttgtggtgatgccagtagaatggctagagatgttcaagctgcaattgccaagattgttgccaaaagtagagatatccacgaagataaagctgctgaattggttaaatcttggaaagttcaaaatagataccaagaagatgtctggtaa

SEQ ID NO: 4 - DNA, 4296 bp, Candida tropicalis
tgcatactcggagcatatcgccatcgtccatatcgttggcactccatccactgagtcagccaagaagcaattgttgttgcaccacaccttaggtaatggtgactttactgttttccacaagatctcgtcattcatcagtgccactactgctgggttgaccgacccagacaccgccgctgatgaaattgatagagtgattgagtcagcctacatcaaccagcgtccaacgtacttgggattcccttccaacatggttgacgttcaagtgccagtcagcaagttggacaagccattgaacttaaccccacctgcaaacaatccaaagatccagtctgaggtcttgagcgacattattgccttgattgaaaccgccaaggatccagttatcatcattgatgcttgttgtggaaggcacaatgctaccccagaggcacagaagttgattgagttgacaaagttcaagtttgctgtcaccccaatggctaaagggtctaaggacattgatgaaagtgatccgaagttcattggttgctacgttggtgacttgtcttatccaagagtcaaagagttggttgaaagctcggacttggtcttgtccttgggtgctgtcttgtctgatttcaacactggttcgttctcatactctttggacaatgccaaggttgttgaattccactccgactacactcaaatcaagagcgctcagtacccaggtatcagaatgaaggaattgttgggcaagttggttgaggagccagaattggtcaagacgtgttccaagatcccagcaaagaagttggtcactgacaactttgaaccattggtcttgccaccggaccacaagctcacccaatcctggttgtggagtaacttgggtaattggttgaaagaaggtgatgtgattgttaccgaaaccggtacttccaatttcggtattgtccagaccaaattcccaaagaatgctgtcggtatctcgcaagtcttgtggggttccattggctactcggtcggttctgccgctggtgccgttatcgccgccgaggagcttgatcccagccgtagagtcatcttgtttgttggtgacggttctttgcagttgaccgtgcaggaaatctccaccatggccagacacaagaacaacatctacatctttgtcttgaacaacaacggtttcaccattgaaagattgattcacggtccagaagctggttacaacagtattcaagaatgggagaacgctgagttattgaagactttcaaggctaccaactacgagagtttcaccgtcaagactgtcggcgaacttgacaaggtgttcaaggatgaaaagtttgccgtcaacgacaagattagattggttgagatcatgttagacactttcgatgctccagagaacttggttaagcaagctgagagatctgccaacaccaacaagtagagtttgtctatgttttccgtttgccttttctttctagtacgagacgttattgaacgaagtttttatatatctagatctaatacatattccatgtctgttcatttttgacggagtttcataaggtggcagtttctaatcaaaggtccgtcattggcgtcgtggcattggcggctcgcatcaactcgtatgtcaatattttctgttaactccgccagacatacgatcaaaacctacaagcaaaaaaattccacatgctttgtttgagatctccacaaacaacaacggggtaagaaaatcatggggcgattaatcatgccatctttgtaaatttctttgtttcaacatcaccctctttagtcaaaccttcacaggactgtctgctctactttgccacccagttcatatataaattaccaacttccaccgagcaccaccaacacctcaccccactctctcccccccccttttttttccagcttagacacacacttcaaactcgacatggctccatttttgcccgaccaggtcgactacaaacacgtcgacacccttatgttattatgtgacgggatcatccacgaaaccaccgtggacgaaatcaaagacgtcattgcccctgacttccccgccgacaaatacgaggagtacgtcaggacattcaccaaaccctccgaaaccccagggttcagggaaaccgtctacaacaccgtcaacgcaaacaccatggatgcaatccaccagttcattatcttgaccaatgttttgggatcaagggtcttggcaccagctttgaccaactcgttgactcctatcaaggacatgagcttggaagaccgtgaaaagttgttagcctcgtggcgtgactcccctattgctgctaaaaggaagttgttcaggttggtttctacgcttaccttggtcacgttcacgagattggccaatgagttgcatttgaaagccattcattatccaggaagagaagaccgtgaaaaggcttatgaaacccaggagattgacccttttaagtaccagtttttggaaaaaccgaagttttacggcgctgagttgtacttgccagatattgatgtgatcattattggatctggggccggtgctggtgtcgtggcccacactttgaccaacgacggcttcaagagtttggttttggaaaagggcagatactttagcaactccgagttgaactttgatgacaaggacggggttcaagaattataccaaagtggaggtactttgaccaccgtcaaccagcagttgtttgttcttgctggttccacttttggtggtggtaccactgtcaattggtcggcctgtcttaaaacgccattcaaggtgcgtaaggaatggtatgatgagtttggcgttgactttgctgccgatgaagcctacgacaaagcacaggattatgtttggcagcaaatgggagcttctaccgaaggcatcacccactctttggctaacgagattattattgaaggtggcaagaaattaggttacaaggccaaggtattagaccaaaacagcggtggtcatcctcatcacagatgcggtttctgttatttgggttgtaagcacggtatcaagcagggctctgttaataactggtttagagacgcagctgcccacggttctcagttcatgcaacaggttagagttttgcaaatccttaacaagaagggcatcgcttatggtatcttgtgtgaggatgttgtaaccggtgccaagttcaccattactggccccaaaaagtttgttgttgccgccggcgccttaaacactccatctgtgttggtcaactccggattcaagaacaagaacatcggtaagaacttaactttgcatccagtttctgtcgtgtttggtgattttggcaaagacgttcaagcagatcacttccacaactccatcatgactgctctttgttcagaagccgctgatttagacggcaagggtcatggatgcagaattgaaaccatcttgaacgctccattcatccaggcttcattcttaccatggagaggtagtaacgaggctagacgagacttgttgcgttacaacaacatggtggccatgttacttcttagtcgtgataccaccagtggttccgtttcgtcccatccaactaaacctgaagcattagttgtcgagtacgacgtgaacaagtttgacagaaactccatcttgcaggcattgttggtcactgctgacttgttgtacattcaaggtgccaagagaatccttagtccccaaccatgggtgccaatttttgaatccgacaagccaaaggataagagatcaatcaaggacgaggactatgtcgaatggagagccaaggttgccaagattccttttgacacctacggctcgccttatggttcggcgcatcaaatgtcttcttgtcgtatgtcaggtaagggtcctaaatacggtgctgttgataccgatggtagattgtttgaatgttcgaatgtttatgttgctgacgctagtcttttgccaactgctagcggtgctaatcctatggtcaccaccatgactcttgcaagacatgttgcgttaggtttggcagactccttgaagaccaaggccaagttgtagttctgtatacgtatcttataatttagatttccttttattgacggtaaacattcaggataggtactacccttgctgcaaaagcccagcacgccccaatcgcgatgacttgagcgaagcaaacacgcacacaaaaggggtacacaaaaaataacgagatgcccttgaagcacacacccaaacacgatggaacacaagatggccctagaaagtacaaaaaaagtaaagccacttgattccgccca

SEQ ID NO: 5 - DNA, 1653 bp, Candida tropicalis
atgacaccaccttctaaaattgaggacagttcatcatcatcatcctcatcagcagattcttccacttttgttactggtaattcatggtatactaaagtatcagatattaaaccaggtgttgatagattaattgacgcttttcatactaaacaaaagactcaagatgttcaatttcgtttaaatcaattgagaaacctttattatgccgttcaagataatactgatgaattatgtgaagctttagagaaagattttcatcgtgcaaatactgaaactaaaaatttagaaattgttggtggattagctgaattagttcatactatgtcaagtttacacgaatggttgaaacctgaaaaagttactgatttaccaattactttaaaaactaatccaatttatattgaaagaattccattaggtgttgttttaattataagtccatttaattatcctttctttttatcattttctgctattgttggtgcaattgctggtggtaatgcagttgttttaaaacaatctgaattagctcctaatttttcaagtttatttagtaaaattttaactaatgctttagatccagatattttcttttctgttgatggtggaattcctgaaattactgaattattagaacaaaaatttgataaaattatgtatactggtaataatactgttggtaaaattattgctaaaaaagccgctgaaactttaactccagttattttagaattaggaggaaaatcacctgcttttattttagatgatgttaaagataaagatttagaagttattgctagaagaattgcatggggtagatttaccaatgctggtcaaacttgtgttgctgttgattatgttttagtttgtgaaaaacttcataaaaaatttgttgatattttaattaaaattttacatgaagaattttatcctggtttaaataaagatactaaaggttatactcatattattcatgatcgagcatttaacaatttatcaaagattatttcaaccacaaaaggagatgttgtatttggaggagaaacagatgctaattcaagatttatttctccaactgttattgataatgcatcatgggatgattcatcaatgaaaggagaaatttttggtccaattttaccaattttatcatatgataaattatcagatgctattaaacaagttttaaatcaacatgatactccattagctcaatatattttcacatctggttcaacttcacgtaaatataatcgtcaattggatcaaattttaacttatgttagatcaggtggggttattatcaatgatgttttaatgcatgttgctttaattaatgctccatttggtggtattggtaattctggttatggttcttatcatggtaaattttcttttagaagttttactcatgaacgtactacaatggaacaaaaattatggaatgataatatggttaaagtaagatatcctccttataattcaaataaagataaattgattaaaatttctcaacaaaatttcaatggtaaagtttggtttgatagaaatggtgatgttccagttaatggaccaagtgggttatttactacatggacttcaattactggtgtctttagtttacttgctgaattcataaccaataaacaataa

SEQ ID NO: 6 - DNA, 53 bp, Artificial Sequence
  LIP1_F
aaactcgagatgagatttcttgtattcattacaattattacatggttgaaaac

SEQ ID NO: 7 - DNA, 61 bp, Artificial Sequence
  LIP1_R
aaatctagagtggtggtggtggtggtggacaagataggtactattcttcacagtgaagctt

SEQ ID NO: 8 - DNA, 42 bp, Artificial Sequence
  CYP52B1_F
aaacccgggatgatcgaacaagttgttgaatactggtacgtg

SEQ ID NO: 9 - DNA, 62 bp, Artificial Sequence
  CYP52B1_R
aaagtcgacgtggtggtggtggtggtgatcgatcttgacaatagttccgtcttgtaaagaca

SEQ ID NO: 10 - DNA, 54 bp, Artificial Sequence
  NCP1_F
aaagtcgacatggcattagataagttagatttatatgttattataacattggtg

SEQ ID NO: 11 - DNA, 62 bp, Artificial Sequence
  NCP1_R
aaactcgaggtggtggtggtggtggtgccagacatcttcttggtatctattttgaactttcc

SEQ ID NO: 12 - DNA, 35 bp, Artificial Sequence
  FAO1_F
aaactcgagatggctccatttttgcccgaccaggt

SEQ ID NO: 13 - DNA, 55 bp, Artificial Sequence
  FAO1_R
aaatctagagtggtggtggtggtggtgcaacttggccttggtcttcaaggagtct

SEQ ID NO: 14 - DNA, 42 bp, Artificial Sequence
  ALD1_F
aaactcgagatgacaccaccttctaaaattgaggacagttca

SEQ ID NO: 15 - DNA, 61 bp, Artificial Sequence; identical to SEQ ID NO: 7
  ALD1_R

(19) United States
(12) Patent Application Publication
(10) Pub. No.: US 2022/0049231 A1
(43) Pub. Date: Feb. 17, 2022

(54) DICARBOXYLIC ACID SYNTHESIS-RELATED ENZYME, AND METHOD FOR PRODUCING DICARBOXYLIC ACID USING SAME

(71) Applicant: Korea University Research and Business Foundation, Seoul (KR)

(72) Inventors: Kyoung Heon KIM, Seoul (KR); Thirumalaisamy BABU, Seoul (KR); Do Hyoung KIM, Yongin-si (KR)

(73) Assignee: Korea University Research and Business Foundation, Seoul (KR)

(21) Appl. No.: 17/299,206

(22) PCT Filed: Dec. 4, 2019
(86) PCT No.: PCT/KR2019/017017

§ 371 Date: Jun. 2, 2021

(30) Foreign Application Priority Data
Dec. 4, 2018 (KR) 10-2018I0154373

(51) Int. Cl. C12N 9/20; C12N 9/02; C12N 9/04; C12P 7/44

(52) U.S. Cl. CPC C12N 9/20; C12Y 301/01003; C12N 9/0079; C12Y 106/02004; C12P 7/44; C12Y 101/0302; C12N 9/0006; C12Y 102/01003; C12N 9/0008; C12N 9/0042

(57) ABSTRACT

The present invention elates to a dicarboxylic acid synthesis-related enzyme, a gene coding for same, and a method for producing dicarboxylic acid using same. The gene or enzyme encoded by the gene of the present invention can be used in bio-enzymatic production, instead of the existing chemical production, of dicarboxylic acid, and is thus expected to have high industrial utility.

### TECHNICAL FIELD

The present invention relates to an enzyme involved in the production of a dicarboxylic acid (DCA), a gene encoding the same, a vector including the gene, and a method for producing a dicarboxylic acid using the same.

### BACKGROUND ART

Dicarboxylic acids (DCAs) are organic compounds containing two carboxyl groups (—COOH). The general molecular formula of dicarboxylic acids may be represented by HO₂C—R—CO₂H, wherein R may be an aliphatic or aromatic group. In general, dicarboxylic acids exhibit chemical reactions and reactivity similar to monocarboxylic acids. Dicarboxylic acids are also used to prepare copolymers such as polyamides and polyesters. The most widely used dicarboxylic acid in the industry is adipic acid, which is a precursor used in the production of nylon. Other examples of dicarboxylic acids include aspartic acid and glutamic acid, which are two amino acids in the human body. In addition, other carboxylic acids have been used in various industries fields.

Such dicarboxylic acids have been prepared by chemical processes or biological methods. As one example regarding the preparation of dicarboxylic acids, the synthesis of sebacic acid, which is one of the dicarboxylic acids, is possible even using phenol and cresol, but castor oil oxidation is known to be the most environmentally friendly and price-competitive method. Castor oil is transesterified by means of steam cracking, and ricinoleic acid is produced through the transesterification. When the ricinoleic acid thus produced is heated at 250° C. and then mixed with an alkali such as molten caustic soda, and the like, the ricinoleic acid is decomposed into capryl alcohol (2-octanol) and sebacic acid by means of caustic digestion. The product thus produced is purified to yield high-purity sebacic acid (U.S. Pat. Nos. 5,952,517 and 6,392,074). However, such a method has a drawback in that it requires a high-temperature process performed at 300° C. or higher to achieve the above, strong acids such as sulfuric acid are used, and large amounts of environmental contaminants are produced as substances such as heavy metals, toxic organic solvents, and the like are used therein. Such production is also possible by electrolyzing potassium monoethyl adipate in addition to using a chemical method for preparing sebacic acid.

In previous studies, cases of biologically producing dicarboxylic acids using a Candida tropicalis strain, which has excellent w-oxidation capacity and whose β-oxidation is blocked, have been reported. However, there is no report regarding a specific dicarboxylic acid biosynthesis pathway. Therefore, it is important to identify a dicarboxylic acid biosynthesis pathway to develop a useful strain capable of mass-producing dicarboxylic acids.

Accordingly, the present inventors have found that genes associated with the dicarboxylic acid biosynthesis are screened by an evolutionary method using a Candida tropicalis strain producing the dicarboxylic acids, and a biosynthesis pathway is identified using the genes. Therefore, the present invention has been completed on these facts.

### RELATED-ART DOCUMENT

### Patent Document

Korean Patent Application No. 10-2015-0149253

### Non-Patent Document

David L. Craft, et al., Applied and Environmental Microbiology, 69 (10): 5983-5991, 2003

### DISCLOSURE

### Technical Problem

Therefore, it is an object of the present invention to provide a protein involved in the biosynthesis of a dicarboxylic acid (DCA), which includes one or more selected from a lipase (LIP1), cytochrome P450 52B1 (CYP52B1), an NADPH-cytochrome P450 reductase (NCP1), a long-chain alcohol oxidase (FAO1), and an aldehyde dehydrogenase (ALD1).

It is another object of the present invention to provide a recombinant vector including a gene encoding the protein, and a composition for producing a dicarboxylic acid, which includes the recombinant vector.

It is still another object of the present invention to provide a method for producing a dicarboxylic acid, which includes incubating a microorganism transformed with the vector including the gene.

### Technical Solution

In previous studies, the production of dicarboxylic acids has been performed by a chemical method and most strains known to produce dicarboxylic acids die because the strains are vulnerable to the cytotoxicity of substrates. Therefore, it was not easy to produce dicarboxylic acids by a biological method or identify a dicarboxylic acid production pathway. Accordingly, the present inventors have manufactured a strain, which has high tolerance to cytotoxic substrates and produces dicarboxylic acids, using an evolutionary method in a previous study, and screened enzymes associated with the dicarboxylic acid biosynthesis and genes encoding the enzymes from the strain. Therefore, the present invention has been completed on these facts.

Particularly, the present invention provides a protein involved in the biosynthesis of a dicarboxylic acid (DCA), which includes one or more selected from a lipase (LIP1), cytochrome P450 52B1 (CYP52B1), an NADPH-cytochrome P450 reductase (NCP1), a long-chain alcohol oxidase (FAO1), and an aldehyde dehydrogenase (ALD1).

The proteins may be derived from a Candida tropicalis strain, but the present invention is not particularly limited thereto.

According to one embodiment, Candida tropicalis strains known as strains producing sebacic acid, which is one of the dicarboxylic acids, are incubated in a medium containing a substrate exhibiting cytotoxicity to screen the strains having excellent ability to survive in the substrate in an evolutionary aspect, and a lipase gene, a cytochrome P450 52B1 (CYP52B1) gene, an NADPH-cytochrome P450 reductase (NCP1) gene, a long-chain alcohol oxidase gene, and an aldehyde dehydrogenase gene, which are represented by base sequences set forth in SEQ ID NOs: 1 to 5, respectively, are selected as endogenous genes, which are estimated to be associated with dicarboxylic acid metabolism, through the genome analysis of the screened strains. As a result, it is confirmed that the enzymes expressed from the genes produce dicarboxylic acids when the enzymes enzymatically react in vitro with a substrate.

The lipase may be expressed by the gene set forth in SEQ ID NO: 1, the cytochrome P450 52B1 (CYP52B1) may be expressed by the gene set forth in SEQ ID NO: 2, the NADPH-cytochrome P450 reductase (NCP1) may be expressed by the gene set forth in SEQ ID NO: 3, the long-chain alcohol oxidase may be expressed by the gene set forth in SEQ ID NO: 4, and the aldehyde dehydrogenase may be expressed by the gene set forth in SEQ ID NO: 5.

Also, the genes, which are represented by the base sequences set forth in SEQ ID NOs: 1 to 5, respectively, are genes that include one or more mutations such as substitutions, deletions, translocations, additions, and the like. In this case, each of the enzymes expressed from the genes also include genes having enzymatic activities of the lipase, the cytochrome P450 52B1, the NADPH-cytochrome P450 reductase, the long-chain alcohol oxidase, and the aldehyde dehydrogenase. Particularly, each of the enzymes includes a base sequence having a sequence homology of 80% or more, 85% or more, 90% or more, 93% or more, 94% or more, 95% or more, 96% or more, 97% or more, 98% or more, and 99% or more to one of the base sequences set forth in SEQ ID NOs: 1 to 5.

One or more of the genes may be included in a vector. The vector may be in a form in which genes can be operably linked. In the present invention, the term “operably linked” generally means that a base-expressing regulatory sequence is operably linked to a base sequence encoding a desired protein to perform its function, thereby exerting an influence on the expression of the base sequence encoding the desired protein. The operable linking of the vector may be achieved using genetic recombination techniques known in the art, and site-specific DNA digestion and ligation may be performed using digestion and ligation enzymes and the like known in the art.

In the present invention, the term “vector” refers to any medium for cloning and/or transferring bases into a host cell. The vector may be a replicon that may bind to another DNA fragment to replicate the bound fragment. The term “replicon” refers to any genetic unit (for example, a plasmid, a phage, a cosmid, a chromosome, a virus) that functions in vivo as an autologous unit of DNA replication, that is, is replicable through the its own regulation. The term “vector” may include viral and non-viral mediums for introducing bases into a host cell in vitro, ex vivo, or in vivo. Also, the term “vector” may include mini-spherical DNA. For example, the vector may be a plasmid that does not have a bacterial DNA sequence. The term “vector” may also include a transposon such as Sleeping Beauty (Izsvak et. al. J. Mol. Biol. 302:93-102 (2000)), or an artificial chromosome. Examples of commonly used vectors include naturally occurring or recombinant plasmids, cosmids, viruses, and bacteriophages. For example, pWE15, M13, MBL3, MBL4, IXII, ASHII, APII, t10, t11, Charon4A, Charon21A, and the like may be used as the phage vector or the cosmid vector. A plasmid vector may also be used. Vectors that may be used in the present invention are not particularly limited, and known expression vectors may be used. Preferably, vectors overexpressing the genes may be used.

The present invention provides a composition for producing a dicarboxylic acid, which includes one or more of the above-described five proteins, and also provides a composition for producing a dicarboxylic acid, which includes a recombinant vector including one or more of the genes set forth in SEQ ID NOs: 1 to 5, which encode the proteins, respectively.

The present invention provides a recombinant vector including one or more of the genes, and a microorganism having an ability to produce dicarboxylic acid, which is transformed with a composition including the recombinant vector.

For example, the microorganism may be algae, a virus, a bacterium, yeast, and a fungus. More particularly, the microorganism may be a Candida tropicalis strain. The Candida tropicalis strain is a strain whose β-oxidation pathway is blocked. Particularly, the Candida tropicalis strain is a strain whose β-oxidation pathway is blocked, thereby producing dicarboxylic acids using a substrate.

Also, the present invention provides a method for producing a dicarboxylic acid, which includes incubating one or more proteins selected from a lipase (LIP1), cytochrome P450 52B1 (CYP52B1), an NADPH-cytochrome P450 reductase (NCP1), a long-chain alcohol oxidase (FAO1), and an aldehyde dehydrogenase (ALD1) with a substrate.

According to specific embodiments of the present invention, the method may be a method for producing a dicarboxylic acid, which includes:

(1) enzymatically reacting the lipase (LIP1) with a C₆-C₂₀ fatty acid methyl ester;

(2) enzymatically reacting the product of the step (1) with the cytochrome P450 52B1 (CYP52B1) and the NADPH-cytochrome P450 reductase (NCP1);

(3) enzymatically reacting the product of the step (2) with the long-chain alcohol oxidase (FAO1); and

(4) enzymatically reacting the product of the step (3) with the aldehyde dehydrogenase (ALD1).

The method for enzymatically producing a dicarboxylic acid according to the present invention may be performed in vitro, and the time-sequential enzymatic reaction step may be considered to be a new pathway for biosynthesis of dicarboxylic acids.

According to still another aspect, the present invention provides a method for producing a dicarboxylic acid (DCA), which includes incubating a microorganism, which is transformed with a vector including a gene encoding the protein, with a substrate.

The method for producing a dicarboxylic acid according to the present invention uses the above-described lipase (LIP1), cytochrome P450 52B1 (CYP52B1), NADPH-cytochrome P450 reductase (NCP1), long-chain alcohol oxidase (FAO1), and aldehyde dehydrogenase (ALD1), or the genes encoding the proteins, as they are. Therefore, a description of the common contents between the two is omitted to avoid excessive complexity of this specification.

The substrate used in the method for producing a dicarboxylic acid may be a fatty acid methyl ester (FAME). Particularly, the fatty acid methyl ester may be one of fatty acid methyl esters including a C₆-C₂₀ alkylene group. More particularly, the fatty acid methyl ester may be decanoic acid methyl ester (DAME).

Microorganisms transformed with the vector including the genes are not limited, but the Candida tropicalis strain may preferably be a strain whose β-oxidation pathway is blocked.

### Advantageous Effects

It has been confirmed that the genes obtained according to the present invention are associated with the production of dicarboxylic acids. Also, it has been found that enzymes expressed by the genes exhibit the activity of producing precursor materials of dicarboxylic acids. Therefore, this is applicable to a process for enzymatically or biologically producing a dicarboxylic acid, which overcomes the drawbacks of existing chemical dicarboxylic acid production processes and is more environmentally friendly and safer, and is thus expected to have high industrial utility.

### DESCRIPTION OF DRAWINGS

FIG. 1 is a schematic diagram showing a biosynthesis pathway for sebacic acid, which is one of the dicarboxylic acids, and genes associated with the biosynthesis pathway.

FIG. 2 shows the results of GC/MS analysis of in vitro reaction products of a Lip1p enzyme.

FIG. 3 shows the results of GC/MS analysis of in vitro reaction products of Cyp52B1p and Ncp1p enzymes.

FIG. 4 shows the results of GC/MS analysis of in vitro reaction products of Fao1p and Ald1p enzymes.

### BEST MODE

Hereinafter, the constitution and the effects of the present invention will be described in further detail with reference to embodiments thereof. However, it should be understood that the embodiments described herein are merely provided for exemplary illustration of the present invention, and are not intended to limit the scope of the present invention.

### [Example 1] Development of DAME-Tolerant Strain Using Evolutionary Engineering Method

To develop a strain having tolerance to DAME, which is a substrate having cytotoxicity, a C. tropicalis MYA_3404 strain was incubated in a YNB medium (10 g/L of a yeast extract and 20 g/L of peptone) to which DAME was added at a concentration of 10 g/L. In this case, it was confirmed that the concentration of DAME in the medium was maintained to be approximately 0.45 g/L (maximal solubility) due to the low solubility of the DAME substrate (confirmed through the results of internal experiments). The growth curve of the inoculated strain was determined by measuring an absorbance value at a wavelength of 600 nm.

The absorbance of the medium in which the strain was inoculated was observed in real time, and the strain was then sub-cultured in a fresh medium until the growth of the strain reached a mid-exponential phase. A specific growth rate of the strain was calculated from the measured absorbance value, and strains having phases where a specific growth rate was greatly changed were determined to be E1 (170 generation time), E2 (470 generation time), E3 (650 generation time), E4 (700 generation time), and E5 (720 generation time), respectively. Also, the E5 strain obtained by the method as described above was sub-cultured in a YNB medium (10 g/L of a yeast extract and 20 g/L of peptone) supplemented with 20 g/L of glucose as a non-toxic carbon source, and then re-incubated in a DAME substrate to screen a strain whose tolerance to DAME was maintained even after replacing the carbon source, which was named “ES5.”

### [Example 2] Transcriptome Analysis of DAME-Tolerant Mutant Strain (ES5)

To check a change of a transcriptome in media with and without DAME, the transcriptomes of the ES5 strain grown in a medium supplemented with DAME and the ES5 strain grown in a DAME-free medium were analyzed.

The ES5 strains were incubated in a DAME-free YNB medium and a YNB medium supplemented with 10 g/L of DAME at 30° C. for 24 hours. The incubated cells were collected, and washed with water. Thereafter, the collected cells were used as a sample for whole RNA extraction. The RNA extraction was performed using an RNeasy Mini Kit (Qiagen, Hilden, Germany), and the concentration and purity of the extracted RNA were measured using NanoDrop (Thermo Scientific, Wilmington, Del., USA) and Agilent Bioanalyzer 2100 (Santa Clara, Ca, USA), respectively.

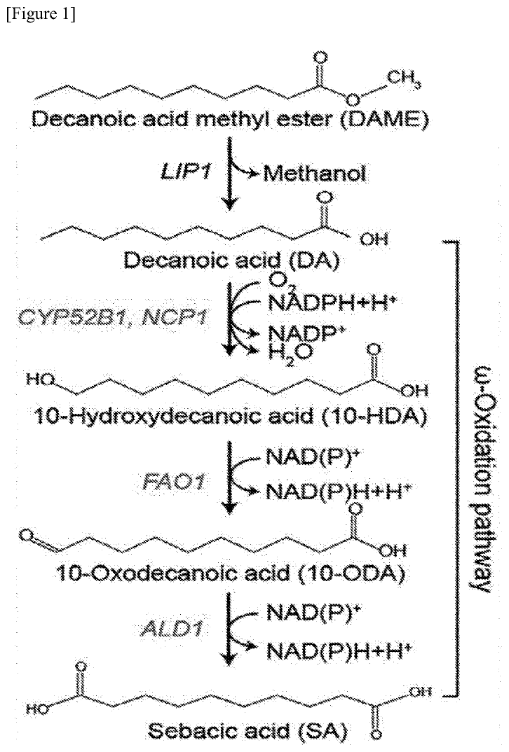

The transcriptome of the mutant ES5 strain was analyzed, and compared with that of the parent strain. As a result, it was confirmed that a total of 453 genes were upregulated in the ES5 strain, compared to the parent strain, and 147 genes were downregulated in the ES5 strain, compared to the parent strain. The details of the number and clusters of the genes are specified in Table 1. The five genes (LIP1, CYP52B1, NCP1, FAO1, and ALD1), which were expected to be associated with the metabolism of sebacic acid, which was one of the dicarboxylic acids, among the 453 genes confirmed to be upregulated through the transcriptome analysis, were selected (FIG. 1).

TABLE 1
Results of comparison/analysis of transcriptomes of parent
strain and DAME-tolerant mutant strain (ESS)

| | | No of | No of |
|---|---|---|---|
| | | Upregulated | Downregulated |
| No | Pathway | Genes | Genes |
| 1 | Alanine, aspartate, and glutamate | 12 | |
| | metabolisms | | |
| 2 | alpha-Linolenic acid metabolisms | 9 | |
| 3 | Arginine and proline metabolisms | 12 | |
| 4 | Arginine biosynthesis | 9 | |
| 5 | Ascorbate and aldarate metabolisms | 6 | |
| 6 | Beta-Alanine metabolism | 15 | 6 |
| 7 | Biosynthesis of antibiotics | 51 | |
| 8 | Biosynthesis of unsaturated fatty | 12 | |
| | acids | | |
| 9 | Biotin metabolism | 6 | |
| 10 | Butanoate metabolism | 9 | 6 |
| 11 | Cell cycle - yeast | | 12 |
| 12 | Cysteine and methionine | 12 | |
| | metabolisms | | |
| 13 | DNA replication | | 15 |
| 14 | Fatty acid degradation | | 6 |
| 15 | Fatty acid metabolism | 18 | 6 |
| 16 | Galactose metabolism | | 6 |
| 17 | Glycerolipid metabolism | 12 | |
| 18 | Histidine metabolism | 12 | |
| 19 | Homologous recombination | | 9 |
| 20 | Lysine biosynthesis | 9 | |
| 21 | Lysine degradation | 12 | |
| 22 | Meiosis - yeast | | 15 |
| 23 | Metabolic pathways | 120 | 27 |
| 24 | Mismatch repair | | 6 |
| 25 | Monobactam biosynthesis | 6 | |
| 26 | Nucleotide excision repair | | 6 |
| 27 | Pantothenate and CoA biosynthesis | 12 | |
| 28 | Pentose and glucuronate | | 6 |
| | interconversions | | |
| 29 | Peroxisome | 27 | 6 |
| 30 | Pyruvate metabolism | 18 | |
| 31 | Starch and sucrose metabolisms | | 9 |
| 32 | Steroid biosynthesis | 9 | |
| 33 | Tryptophan metabolism | 9 | |
| 34 | Ubiquinone and other terpenoid- | 9 | |
| | quinone biosynthesis | | |
| 35 | Valine, leucine and isoleucine | 12 | |
| | biosynthesis | | |
| 36 | Valine, leucine and isoleucine | 15 | 6 |
| | degradation | | |
| | Total | 453 | 147 |

### [Example 3] Attainment of SA Biosynthesis Pathway-Related Genes Using Cloning Technique

Based on the results of transcriptome analysis in Example 2, the five genes (LIP1, CYP52B1, NCP1, FAO1, and ALD1) expected to be associated with the sebacic acid biosynthesis pathway were selected, and LIP1 (Uniprot. ID: C5MD87), CYP52B1 (Uniprot. ID: C5MAM3), NCP1 (Uniprot. ID: C5M346), NADPH-cytochrome P450 reductase, FAO1 (Uniprot. ID: Q6QIR6), and ALD1 (Uniprot. ID: C5MEH8) genes were obtained by cloning in order to check the activities of the enzymes (a lipase, cytochrome P450 52B1, a long-chain alcohol oxidase, and an aldehyde dehydrogenase) derived from the five genes. The CYP450 gene is known to have two subunits, CYP1 and NCP1.

The C. tropicalis MYA_3404 strain was incubated at 30° C. for 48 hours in a YPD medium (10 g/L of a yeast extract, 20 g/L of peptone, and 20 g/L of glucose), and template DNA used for cloning was then extracted using a yeast DNA isolation kit (Epicentre, Madison, Wis., USA). A candidate gene was amplified using a Q5 High-Fidelity Master mix (BioLabs, Ipswich, Mass., USA), and the primers used to amplify the candidate gene are as listed in Table 2 (primers 1 to 10; SEQ ID NOs: 6 to 15). Thereafter, PCR was performed in all the experiments for genetic recombination using the same enzymes. A base sequence of a gene encoding a histidine residue was added to enhance the affinity of a HisTrap column. The remaining PCR products other than the CYP450 gene, and a pAUR123 vector was doubly digested with XhoI and XbaI restriction enzymes, and the final DNA fragments were ligated into the same restriction enzyme sites using a T4 DNA ligase (New England Biolabs). As two subunits encoding the CYP450 gene, CYP52B1 (Uniprot. ID: C5MAM3) and NCP1 (Uniprot. ID: C5M346) genes were separately digested with SmaI/SalI, SalI/XhoI, and then sequentially ligated. Thereafter, all four ligated plasmids (Plasmids 1 to 4) were independently transformed into E. coli DH5a (Novagen, Cambridge, Mass., USA). To overexpress the proteins encoded by the genes obtained by the above-described process, the four extracted plasmids (Plasmids 1 to 4) were re-transformed into a C. tropicalis strain. The transformation into yeast was performed according to a LiAc/SS carrier DNA/PEG method using a yeast transformation kit (MP Biomedicals, Solon, Ohio, USA). The proteins were spontaneously expressed without any separate inducer by an auto-induction system of the pAUR123 vector serving as the vector used for gene introduction.

TABLE 2
List of Primeres used to clone SA
biosynthesis-related genes

| | | |
|---|---|---|
| [symbol] | Primers | 5′-3′ sequence |

Seabcic acid pathway related genes

| | | |
|---|---|---|
| 6 | LIP1_F | AAACTCGAGATGAGATTTCTTGTATTCATTACAAT |
| | | TATTACATGGTTGAAAAC (Xhol) |
| 7 | LIP1_R | AAATCTAGAGTGGTGGTGGTGGTGGTGGACAAGAT |
| | | AGGTACTATTCTTCACAGTGAAGCTT (Xbal) |
| 8 | CYP1_F | AAACCCGGGATGATCGAACAAGTTGTTGAATACTG |
| | | GTACGTG (Xmal) |
| 9 | CYP1_R | AAAGTGACGTGGTGGTGGTGGTGGTGATCGATCTT |
| | | GACAATAGTTCCGTCTTGTAAAGACA (Sall) |
| 10 | NCP1_F | AAAGTCGACATGGCATTAGATAAGTTAGATTTATA |
| | | TGTTATTATAACATTGGTG (Sall) |
| 11 | NCP2_R | AAACTCGAGGTGGTGGTGGTGGTGGTGCCAGACAT |
| | | CTTCTTGGTATCTATTTTGAACTTTCC (Xhol) |
| 12 | FAO1_F | AAACTCGAGATGGCTCCATTTTTGCCCGACCAGGT |
| | | (Xhol) |
| 13 | FAO1_R | AAATCTAGAGTGGTGGTGGTGGTGGTGCAACTTGG |
| | | CCTTGGTCTTCAAGGAGTCT (Xbal) |
| 14 | ALD1_F | AAACTCGAGATGACACCACCTTCTAAAATTGAGGA |
| | | CAGTTCA (Xhol) |
| 15 | ALD1_R | AAATCTAGAGTGGTGGTGGTGGTGGTGTTGTTTAT |
| | | TGGTTATGAATTCAGCAAGTAAACTAAAGACAC |
| | | (Xbal) |

TABLE 3

| Name [symbol] | Description [symbol] |
|---|---|
| pET21a [symbol] | Escherichia coli expression vector, Amp^{R} [symbol] |
| pAUR123 [symbol] | Low copy number yeast expression vector, AurA^{R} |
| | for yeast, and Amp^{R} for E. coli [symbol] |
| pRS420 [symbol] | High copy number yeast expression vector, |
| | G418R for yeast, and Amp^{R} for E. coli [symbol] |
| plasmid 1 [symbol] | pAUR123::LIP1 [symbol] |
| plasmid 2 [symbol] | pAUR123::CYP52B1::NCP1 [symbol] |
| plasmid 3 [symbol] | pAUR123:FAO1 [symbol] |
| plasmid 4 [symbol] | pAUR123:ALD1 [symbol] |

[symbol]

indicates data missing or illegible when filed

### [Example 4] Overexpression and Purification of Sebacic Acid Biosynthesis Pathway-Related Enzymes

To overexpress the sebacic acid biosynthesis-related enzymes in the recombinant strain obtained in Example 3, the recombinant strain was incubated at 30° C. for 24 hours in a YPD medium (10 g/L of a yeast extract, 20 g/L of peptone, and 20 g/L of glucose) supplemented with 0.2 mg/L of Aureobasidin A. To isolate the expressed proteins, the cells were disrupted with ultrasonic waves, and centrifuged. Then, the supernatant was purified using a HisTrap column (GE Healthcare, Piscataway, USA). The purified proteins were concentrated using an Amicon Ultra Centrifugal filter (Millipore, Billerica, Mass., USA). The molecular weights of the expressed enzymes were confirmed to be 50.6 kDa (for Lip1p), 59.3 kDa (for Cyp1) and 76.7 kDa (for Ncp1) (Cyp450p), 77.8 kDa (for Fao1p), and 61.3 kDa (for Ald1p), as measured by SDS-PAGE. The concentrations of the proteins were measured using a bicinchoninic acid (BCA) protein assay kit (Pierce, Rockford, Ill., USA).

### [Example 5] Confirmation of In Vitro Activities of Sebacic Acid Biosynthesis Pathway-Related Enzymes

To check the activities of the Lip1p, Cyp52B1p, Ncp1, Fao1p, and Ald1p obtained in Example 4, an in vitro enzyme assay was performed using a standard material corresponding to each of the substrates.

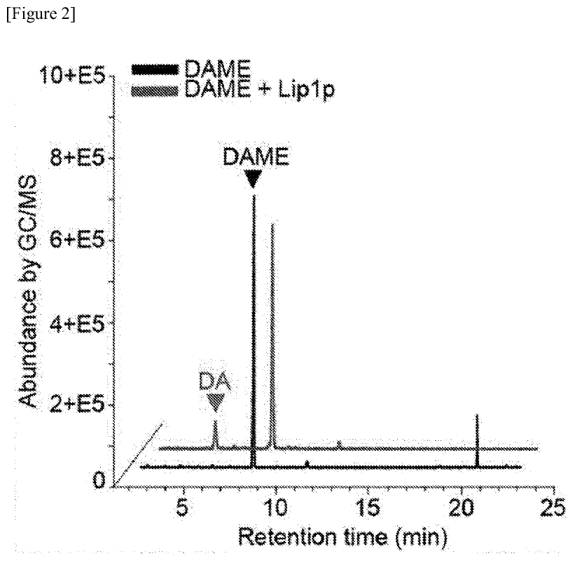

To check the activity of Lip1p, 50 μL of a 100 mM DAME substrate, 250 μL of an enzyme (enzyme concentration: 5 mg/mL), and 200 μL of 20 mM Tris-HCl were reacted, that is, a total of 500 μL of the reaction sample was reacted at 30° C. for an hour. DAME and the inactivated Lip1p enzyme were reacted, and the resulting reaction product was used as the control. As shown in FIG. 2 below, the GC/MS results showed that a peak of DAME decreased while a new peak was generated in the reaction product obtained by reacting the Lip1p enzyme with the substrate. Then, the mass spectrum of this peak was compared with that of the standard material. As a result, it was confirmed that the newly generated peak corresponded to DA.

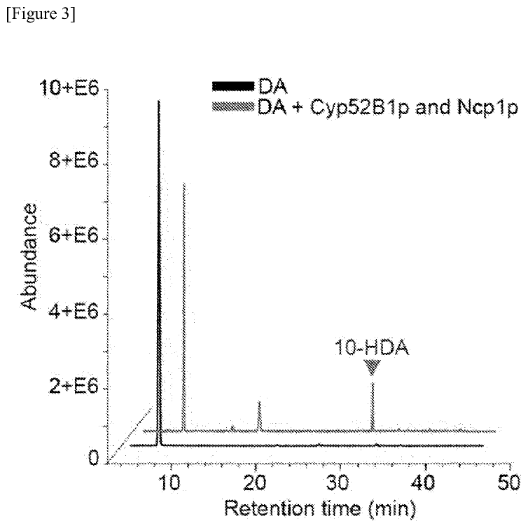

To check the activities of Cyp52B1p and Ncp1p using the same method as described above, 50 μL of a 100 mM DA substrate, 100 μL of an enzyme (enzyme concentration: 2.99 mg/ml), and 50 μL of 20 mM Tris-HCl were reacted, that is, a total of 200 μL of the reaction sample was reacted at 30° C. for an hour. DA and the inactivated Cyp52B1p and Ncp1p enzymes were reacted, and the resulting reaction product was used as the control. As shown in FIG. 3 below, the GC/MS results showed that a peak of DA decreased while a new peak was generated in the reaction product obtained by reacting the Cyp52B1p and Ncp1p enzymes with the substrate. Then, the mass spectrum of this peak was confirmed. As a result, it was confirmed that the newly generated peak corresponded to 10-hydroxydecanoic acid (FIG. 3).

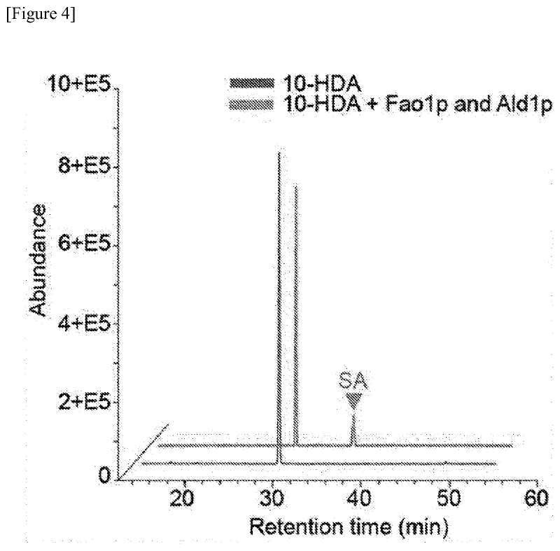

Because the standard material (10-oxodecanoic acid) expected to be the reaction product was not purchased, the activity of the Fao1p enzyme was determined as follows. That is, 10 μL of 100 mM 10-HAD as the substrate, 100 μL of Fao1p (enzyme concentration: 2 0.7 mg/ml), and 100 μL of Ald1p (enzyme concentration: 2.0 mg/ml) as the last biosynthesis-related enzyme were sequentially reacted, and the reaction product was then analyzed. As a result, it was confirmed that SA was produced (FIG. 4).

From the above-described results, the activities of the enzymes associated with the biosynthesis of sebacic acid, which is one of the dicarboxylic acids, were determined, and a novel biosynthesis pathway for dicarboxylic acid could be predicted based on these results.

1. A protein involved in the biosynthesis of a dicarboxylic acid (DCA) comprising one or more selected from a lipase (LIP1), cytochrome P450 52B1 (CYP52B1), an NADPH-cytochrome P450 reductase (NCP1), a long-chain alcohol oxidase (FAO1), and an aldehyde dehydrogenase (ALD1).

2. The protein of claim 1, wherein the proteins are derived from a Candida tropicalis strain.

3. The protein of claim 1, wherein the dicarboxylic acid is a C6-C20 dicarboxylic acid.

4. The protein of claim 1, wherein the lipase (LIP1) is expressed by a gene set forth in SEQ ID NO: 1; the cytochrome P450 52B1 (CYP52B1) is expressed by a gene set forth in SEQ ID NO: 2; the NADPH-cytochrome P450 reductase (NCP1) is expressed by a gene set forth in SEQ ID NO: 3; the long-chain alcohol oxidase (FAO1) is expressed by a gene set forth in SEQ ID NO: 4; and the aldehyde dehydrogenase (ALD1) is expressed by a gene set forth in SEQ ID NO: 5.

5. A composition for biosynthesis of a dicarboxylic acid comprising a recombinant vector comprising one or more genes selected from the group consisting of a gene set forth in SEQ ID NO: 1; a gene set forth in SEQ ID NO: 2; a gene set forth in SEQ ID NO: 3; a gene set forth in SEQ ID NO: 4; and a gene set forth in SEQ ID NO: 5.

6. A microorganism having an ability to produce a dicarboxylic acid (DCA), wherein the microorganism is transformed with the composition defined in claim 5.

7. The microorganism of claim 6, wherein the microorganism is a Candida tropicalis strain whose β-oxidation pathway is blocked.

8. A method for producing a dicarboxylic acid (DCA), the method comprising:
incubating the protein defined in claim 1 with a substrate.

9. The method of claim 8, wherein the substrate is a fatty acid methyl ester (FAME).

10. The method of claim 9, wherein the fatty acid methyl ester substrate comprises one or more selected from C₆-C₂₀ fatty acid methyl esters.

11. The method of claim 8, which comprises:
(1) enzymatically reacting the lipase (LIP1) with a C₆-C₂₀ fatty acid methyl ester;
(2) reacting the product of the step (1) with the cytochrome P450 52B1 (CYP52B1) and the NADPH-cytochrome P450 reductase (NCP1);
(3) enzymatically reacting the product of the step (2) with the long-chain alcohol oxidase (FAO1); and
(4) enzymatically reacting the product of the step (3) with the aldehyde dehydrogenase (ALD1).

12. A method for producing a dicarboxylic acid (DCA), the method comprising:
incubating the microorganism defined in claim 6 with substrate in a medium.

13. The method of claim 12, wherein the microorganism is a Candida tropicalis strain whose β-oxidation pathway is blocked.

14. The method of claim 12, wherein the substrate is a fatty acid methyl ester (FAME).

15. The method of claim 14, wherein the fatty acid methyl ester comprises one or more selected from C₆-C₂₀ fatty acid methyl esters.
